(19) United States
(12) Patent Application Publication
(10) Pub. No.: US 2013/0233333 A1
(43) Pub. Date: Sep. 12, 2013

(54) PROCESS FOR TREATING KERATIN FIBRES USING AT LEAST ONE SULFUREOUS REDUCING AGENT, AT LEAST ONE CATIONIC POLYMER AND AT LEAST ONE MERCAPTOSILOXANE

(71) Applicants: Charlotte Roulet, Paris (FR); Laurence Paul, Saint Leu La Foret (FR); Gaëlle Calonne, Houilles (FR); Aude Livoreil, Paris (FR)

(72) Inventors: Charlotte Roulet, Paris (FR); Laurence Paul, Saint Leu La Foret (FR); Gaëlle Calonne, Houilles (FR); Aude Livoreil, Paris (FR)

(73) Assignee: L'OREAL, Paris (FR)

(21) Appl. No.: 13/821,995

(22) PCT Filed: Sep. 30, 2011
(86) PCT No.: PCT/EP11/67109

§ 371 Date: May 24, 2013

(30) Foreign Application Priority Data
Oct. 1, 2010 (FR) 1057971

(51) Int. Cl. A45D 7/06; A61K 8/899; A61K 8/73; A61K 8/22; A61K 8/898; A61Q 5/04; A61K 8/46

(52) U.S. Cl. CPC A45D 7/06; A61Q 5/04; A61K 8/899; A61K 8/46; A61K 8/22; A61K 8/898; A61K 8/737
USPC 132/204; 424/70.5

(57) ABSTRACT

Process for treating keratin fibres using at least one sulfureous reducing agent, at least one cationic polymer and at least one mercaptosiloxane The present invention relates to a process for treating keratin fibres, in particular human keratin fibres such as the hair, comprising a step of applying to the keratin fibres a reducing composition (A) comprising one or more sulfureous reducing agents, optionally a step of applying to the keratin fibres an oxidizing composition (B), a step of applying a rinse-out or leave-in care composition (C), comprising one or more cationic polymers and optionally one or more amino silicones (i), it being understood that the reducing composition (A) and/or the oxidizing composition (B) and/or composition (C) comprise(s) one or more silicones (ii), other than the silicones (i), with a molecular weight of less than 10 000 and functionalized with one or more mercapto groups.

The present invention relates to a process for treating keratin fibres, in particular human keratin fibres such as the hair, comprising a step of applying to the keratin fibres a reducing composition (A) comprising one or more sulfureous reducing agents, optionally a step of applying to the keratin fibres an oxidizing composition (B), a step of applying a rinse-out or leave-in care composition (C) comprising one or more cationic polymers, and a step of heating the keratin fibres to a temperature ranging from 60 to 250° C. after application of composition (C), it being understood that at least one of the compositions (A), (B) and (C) also comprises one or more silicones (ii) with a molecular weight of less than 10 000 and functionalized with one or more mercapto groups.

The invention also relates to a multi-compartment device or “kit” using the said compositions (A), (B) and (C).

The most common technique for permanently reshaping the hair consists, in a first stage, in opening the —S—S— disulfide bonds of keratin (cystine) using a composition containing a suitable reducing agent (reduction step), and then, after having rinsed the head of hair thus treated, generally with water, in reconstituting the said disulfide bonds, in a second stage, by applying to the hair, which has been placed under tension beforehand (rollers or the like) or shaped or straightened by other means, an oxidizing composition (oxidation step, also known as the fixing step) so as finally to give the hair the desired shape. This technique thus makes it possible, without preference, either to make the hair wavy (permanent-waving process) or to relax or uncurl it (straightening process). The new shape given to the hair by a chemical treatment such as that above is eminently long-lasting and especially withstands washing with water or shampoo, as opposed to simple standard techniques of temporary reshaping, such as hairsetting.

The reducing compositions that may be used for the first step of a permanent-waving operation generally contain sulfites, bisulfites, alkylphosphines or, preferably, thiols as reducing agents. Among the latter, those commonly used are cysteine and derivatives thereof, cysteamine and derivatives thereof, thiolactic acid or thioglycolic acid, and salts thereof and also esters thereof, especially glyceryl thioglycolate.

The oxidizing compositions required for performing the fixing step are usually compositions based on aqueous hydrogen peroxide solution.

In the context of hair relaxing and straightening techniques, this permanent reshaping operation is generally performed on curly or voluminous hair so as to obtain more or less pronounced straightening and a reduction of the volume and apparent mass of the hair.

However, such a technique is not entirely satisfactory. Specifically, although this technique proves to be very effective for modifying the shape of the hair, it still degrades the hair fibres, which is mainly due to the high contents of reducing agents in the reducing compositions and also to the various longer or shorter leave-on times that may be involved in such a process.

Moreover, if the technique of permanent reshaping of the hair described previously is applied to hair that has undergone a prior artificial coloration, it usually leads to degradation or stripping of this artificial coloration.

Similarly, if a coloration is applied to permanent-waved hair according to the technique described previously, the colour obtained is very different from the colour normally obtained on non-permanent-waved natural hair.

In order to overcome all the drawbacks described above, many alternatives have already been envisaged in the implementation of processes for reshaping keratin fibres.

To this end, document FR 2 868 306 describes a process for treating hair fibres without fixing, comprising a step of applying a ceramide-free reducing composition containing at least one thiol-based reducing agent and at least one non-polymeric active agent, and a step of raising the temperature of the fibres by means of a heating iron.

Document FR 2 868 305 describes a process for treating hair fibres without fixing, comprising a step of applying a ceramide-free reducing composition containing at least one thiol-based reducing agent in a content of less than 3% by weight if the reducing composition does not contain any aminothiols, and in a content of less than 5% by weight if the reducing composition contains at least one aminothiol. This process also includes a step of raising the temperature of the hair fibres, which is performed with a heating iron.

Similarly, document FR 2 823 110 describes a process for permanently reshaping keratin fibres, comprising at least the operations consisting in applying to the keratin fibres a reducing composition, in performing oxidation of the keratin fibres and in applying a pretreatment and/or post-treatment composition comprising an amino silicone microemulsion, the number-average primary size of the particles of the microemulsion being between 3 and 70 nm.

For its part, patent application JP2002-356 408 relates to a permanent-waving or straightening process comprising a step of applying a reducing composition comprising a thiol-based reducing agent, a cationic surfactant, a high molecular weight alcohol and an oily compound.

Finally, the unpublished document FR 0 952 475 relates to a process for shaping hair fibres, comprising a step of applying a reducing composition containing at least one cationic polymer, the reducing agent/cationic polymer weight ratio being between 0.1 and 10, a step of applying a care composition comprising an amino silicone, and a step that consists in raising the temperature of the hair fibres to a temperature of between 50 and 280° C.

It has been observed that none of the described processes can lead to shaping of keratin fibres that is entirely satisfactory with regard to the problems mentioned hereinabove.

Moreover, there currently exists in certain countries, and in particular in Brazil, a very strong consumer trend toward haircare treatments based on keratin and formaldehyde. These treatments afford volume control especially in the case of humidity-mediated frizziness, and hair that is straight, well-managed and easy to style, and that feels repaired, and do so in a long-lasting manner.

However, the use of formaldehyde poses a problem due to its toxic nature.

There is thus a real need to develop processes for treating keratin fibres, in particular human keratin fibres such as the hair, that do not have the combination of drawbacks described above, i.e. that do not involve the use of toxic compositions and that are capable of giving the hair volume control and a smooth, soft feel, and of doing so in a long-lasting manner.

The Applicant has discovered, surprisingly, that it is possible to achieve the desired properties by performing a cosmetic process for treating keratin fibres comprising the successive steps of applying to the said fibres a rinse-out reducing composition (A) comprising one or more sulfureous reducing agents, optionally a rinse-out oxidizing composition (B) and a rinse-out or leave-in care composition (C), comprising one or more cationic polymers, and a step of heating the keratin fibres to a temperature ranging from 60 to 250° C.; at least one of the compositions (A) and/or (B) and/or (C) comprising one or more silicones with a molecular weight of less than 10 000 and functionalized with one or more mercapto groups.

The treatment process according to the invention makes the hair smooth, both to the touch and visually, gives it softness and affords gentle curl relaxation.

In addition, the process according to the invention can reduce the frizziness of keratin fibres, which makes the head of hair easier to control.

Moreover, the treatment process according to the invention gives a longer-lasting effect than standard straightening care, the softness possibly being noticeable up to 12 shampoo washes after application.

Moreover, compared with standard straightening, the application of the various compositions (A), (B) and (C) does not require the use of an expert hand and can be performed on keratin fibres that may be damaged, without degrading their cosmetic properties or their colours.

One subject of the present invention is thus especially a cosmetic process for treating keratin fibres, in particular human keratin fibres such as the hair, comprising:

(a) a step of applying to the keratin fibres a reducing composition (A) comprising one or more sulfureous reducing agents, and then

(b) a rinsing step, and then

(c) an optional fixing step by applying to the keratin fibres an oxidizing composition (B) comprising one or more oxidizing agents, this application being followed by rinsing, and then

(d) a step of applying a care composition (C) comprising one or more cationic polymers,

(e) an optional rinsing step, and then

(f) a step of heating the keratin fibres to a temperature ranging from 60 to 250° C. after application of the care composition (C);

it being understood that one or more of the said compositions (A), (B) and (C) also comprise(s) one or more silicones (ii) with a molecular weight of less than 10 000 and functionalized with one or more mercapto groups.

Similarly, the present invention also relates to a multi-compartment device or “kit” comprising the cosmetic compositions (A), (B) and (C) according to the invention.

Other subjects and characteristics, aspects and advantages of the invention will emerge even more clearly on reading the description and the examples that follow.

The reducing composition (A) used for reducing the disulfide bonds of keratin in the process according to the invention comprises one or more sulfureous reducing agents, preferably chosen from the reducing agents of formula:

H(X′)_{q}(R′)ᵣ

in which X′ represents S or SO₂, q is 0 or 1, r is 1 or 2 or 3, and R′ is a linear, branched, saturated or unsaturated C₁-C₂₀ hydrocarbon-based radical, optionally interrupted with a heteroatom, and optionally comprising substituents chosen from a hydroxyl group, a halogenated group, an amine group or a carboxyl group, a (C₁-C₃₀ alkoxy)carbonyl group, an amido group, a (C₁-C₃₀ alkyl)aminocarbonyl group, a (C₁-C₃₀ acyl)amino group, a monoalkylamino or dialkylamino group, or a monohydroxyamino or dihydroxyamino group, or a salt thereof in combination with a base.

The sulfureous reducing agent(s) used in the reducing composition (A) are chosen from thiol-based and non-thiol-based reducing agents.

As thiol-based reducing agents that may be used in the reducing composition (A), mention may be made of thiol-based reducing agents chosen from thioglycolic acid, thiolactic acid, cysteine, homocysteine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiopropionic acid, lipoic acid and N-acetylcysteine, and salts thereof.

As non-thiol-based reducing agents that may be used in the reducing composition (A), mention may be made especially of alkali metal or alkaline-earth metal sulfites.

Preferably, the reducing agent(s) used in the reducing composition (A) are thiol-based reducing agents, in particular thioglycolic acid and thiolactic acid or salts thereof, and even more preferentially thioglycolic acid.

The reducing agent(s) generally represent from 0.1% to 10% by weight, preferably from 0.1% to 4% by weight and better still from 0.3% to 3.3% by weight relative to the total weight of the reducing composition (A).

The reducing composition (A) used in the process according to the invention may also comprise one or more cosmetic active agents.

This or these cosmetic active agent(s) are generally chosen from anionic, cationic, nonionic, amphoteric and zwitterionic surfactants, swelling agents and penetrants, disulfide compounds of reducing agents, for example dithioglycolic acid, thickening polymers of natural or synthetic origin, fixing polymers, conditioning agents such as cationic surfactants, silicones and chitosans, and derivatives thereof, hydrophobic solvents, fatty alcohols, direct dyes, in particular cationic or natural dyes, oxidation dyes, organic or mineral pigments, UV-screening agents, fillers, for example nacres, TiO₂, resins and clays, fragrances, peptizers, vitamins, amino acids, preserving agents and chelating agents.

The surfactants that may be present in the reducing composition (A) used according to the invention are especially the following:

The term “anionic surfactant” means a surfactant comprising, as ionic or ionizable groups, only anionic groups. These anionic groups are preferably chosen from the groups CO₂H, CO₂⁻, SO₃H, SO₃⁻, OSO₃H, OSO₃⁻, O₂PO₂H, O₂PO₂H⁻ and O₂PO₂²⁻.

The anionic surfactant(s) that may be used in the compositions of the invention are chosen in particular from alkyl sulfates, alkyl ether sulfates, alkylamido ether sulfates, alkylaryl polyether sulfates, monoglyceride sulfates, alkylsulfonates, alkylamide sulfonates, alkylarylsulfonates, α-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group.

Some of these compounds may be oxyethylenated and then preferably comprise from 1 to 50 ethylene oxide units.

The salts of C₆₋₂₄ alkyl monoesters and polyglycoside-polycarboxylic acids may be chosen from C₆₋₂₄ alkyl polyglycoside-citrates, C₆₋₂₄ alkyl polyglycoside-tartrates and C₆₋₂₄ alkyl polyglycoside-sulfosuccinates.

When the anionic surfactant(s) (iii) are in salt form, they are not in the form of zinc salts, and they may be chosen from alkali metal salts, such as the sodium or potassium salt, and preferably the sodium salt, ammonium salts, amine salts, and in particular amino alcohol salts, and alkaline-earth metal salts such as the magnesium salt.

Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts, and in particular sodium or magnesium salts, are preferably used.

Use is preferably made of (C₆₋₂₄)alkyl sulfates and (C₆₋₂₄)alkyl ether sulfates, which are optionally oxyethylenated, comprising from 2 to 50 ethylene oxide units, and mixtures thereof, especially in the form of alkali metal salts or alkaline-earth metal salts, ammonium salts or amino alcohol salts. More preferentially, the anionic surfactant(s) are chosen from (C₁₀₋₂₀)alkyl ether sulfates, and in particular sodium lauryl ether sulfate containing 2.2 mol of ethylene oxide.

When they are present, the amount of the anionic surfactant or surfactants varies preferably from 0.1% to 50% by weight, more preferably from 4% to 30% by weight, relative to the total weight of the composition.

Examples of nonionic surfactants that may be used in the cosmetic composition according to the invention are described, for example, in the Handbook of Surfactants by M. R. Porter, published by Blackie & Son (Glasgow and London), 1991, pp. 116-178. They are especially chosen from polyethoxylated, polypropoxylated or polyglycerolated alcohols, α-diols and (C₁₋₂₀)alkylphenols, containing at least one fatty chain comprising, for example, from 8 to 18 carbon atoms, the number of ethylene oxide or propylene oxide groups possibly ranging especially from 2 to 50, and the number of glycerol groups possibly ranging especially from 2 to 30.

Mention may also be made of copolymers of ethylene oxide and propylene oxide, optionally oxyethylenated fatty acid esters of sorbitan, fatty acid esters of sucrose, polyoxyalkylenated fatty acid esters, optionally oxyalkylenated alkylpolyglycosides, alkylglucoside esters, derivatives of N-alkylglucamine and of N-acylmethylglucamine, aldobionamides and amine oxides.

Unless otherwise mentioned, the term “fatty” compound (for example a fatty acid) denotes a compound comprising, in its main chain, at least one saturated or unsaturated alkyl chain containing at least 8 carbon atoms, preferably from 8 to 30 carbon atoms, and even better still from 10 to 22 carbon atoms.

When they are present, the amount of the nonionic surfactant or surfactants varies preferably from 0.01% to 20% by weight, more preferably from 0.2% to 10% by weight, relative to the total weight of the composition.

The amphoteric or zwitterionic surfactant(s) that may be used in the present invention may especially be optionally quaternized, secondary or tertiary aliphatic amine derivatives, in which the aliphatic group is a linear or branched chain containing from 8 to 22 carbon atoms, the said amine derivatives containing at least one anionic group such as, for example, a carboxylate, sulfonate, sulfate, phosphate or phosphonate group. Mention may be made in particular of (C₈-C₂₀)alkylbetaines, sulfobetaines, (C₈-C₂₀)alkylamidoC₃-C₈)alkylbetaines or (C₈-C₂₀)alkylamido(C₆-C₈)alkylsulfobetaines. Among the optionally quaternized secondary or tertiary aliphatic amine derivatives that may be used, as defined above, mention may also be made of the compounds having the respective structures (I) and (II) below:

Ra—CONHCH₂CH₂—N+(Rb)(Rc)(CH₂COO—) (I)

in which:

Ra represents a C₁₀-C₃₀ alkyl or alkenyl group derived from an acid

Ra—COOH, preferably present in hydrolysed coconut oil, a heptyl, nonyl or undecyl group,

Rb represents a β-hydroxyethyl group, and

Rc represents a carboxymethyl group;

and

Ra′—CONHCH₂CH₂—N(B)(B′) (II)

in which:

B represents —CH₂CH₂OX′,

B′ represents —(CH₂)_{z}—Y′, with z=1 or 2,

X′ represents the group —CH₂—COOH, CH₂—COOZ′, —CH₂CH₂—COOH, —CH₂CH₂—COOZ′, or a hydrogen atom,

Y′ represents —COOH, —COOZ′ or the group —CH₂—CHOH—SO₃H or —CH₂—CHOH—SO₃Z′,

Z′ represents an ion derived from an alkali metal or alkaline-earth metal, such as sodium, an ammonium ion or an ion derived from an organic amine,

Ra represents a C₁₀-C₃₀ alkyl or alkenyl group derived from an acid,

Ra′—COOH preferably present in coconut oil or in hydrolysed linseed oil, an alkyl group, especially a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Among the abovementioned amphoteric or zwitterionic surfactants, it is preferred to use (C₈-C₂₀ alkyl)betaines such as cocoylbetaine, and (C₈-C₂₀ alkyl)amido(C₂-C₈ alkyl)betaines such as cocoylamidopropylbetaine, and mixtures thereof. More preferably, the amphoteric or zwitterionic surfactant(s) is (are) chosen from cocoylamidopropylbetaine and cocoylbetaine.

When they are present, the amount of the amphoteric or zwitterionic surfactant or surfactants is preferably in the range from 0.01% to 20% by weight, more preferably from 0.5% to 10% by weight, relative to the total weight of the composition.

The term “cationic surfactant” means a surfactant that is positively charged when it is contained in the composition according to the invention. This surfactant may bear one or more positive permanent charges or may contain one or more cationizable functions within the composition according to the invention.

The cationic surfactant(s) that may be used as conditioning agents according to the present invention are preferably chosen from optionally polyoxyalkylenated primary, secondary or tertiary fatty amines, or salts thereof, quaternary ammonium salts, and mixtures thereof.

The fatty amines generally comprise at least one C₈-C₃₀ hydrocarbon-based chain. Among the fatty amines that may be used according to the invention, examples that may be mentioned include stearylamidopropyldimethylamine and distearylamine.

Examples of quaternary ammonium salts that may especially be mentioned include:
those corresponding to the general formula (III) below:

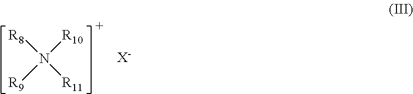

in which the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ denoting a group containing from 8 to 30 carbon atoms, preferably from 12 to 24 carbon atoms. The aliphatic groups can comprise heteroatoms such as, in particular, oxygen, nitrogen, sulfur and halogens. The aliphatic groups are chosen, for example, from C₁₋₃₀ alkyl, C₁₋₃₀ alkoxy, polyoxyalkylene (C₂-C₆), C₁₋₃₀ alkylamide, (C₁₂-C₂₂)alkylamido(C₂-C₆)alkyl, (C₁₂-C₂₂)alkylacetate, and C₁₋₃₀ hydroxyalkyl; X⁻ is an anion chosen from the group of halides, phosphates, acetates, lactates, (C₁-C₄)alkyl sulfates and (C₁-C₄)alkyl sulfonates or (C₁-C₄)alkylaryl sulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethyl-ammonium, cetyltrimethylammonium or benzyldimethylstearyl-ammonium salts, or, on the other hand, the palmitylamidopropyl-trimethylammonium salt, the stearamidopropyltrimethylammonium salt, the stearamidopropyldimethylcetearylammonium salt, or the stearamidopropyldimethyl(myristyl acetate)ammonium salt sold under the name Ceraphyl® 70 by the company Van Dyk. It is particularly preferred to use the chloride salts of these compounds;
quaternary ammonium salts of imidazoline, such as, for example, those of formula (IV) below:

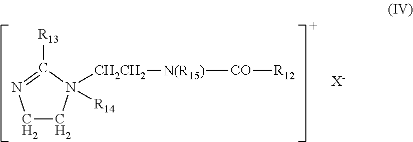

in which R₁₂ represents an alkenyl or alkyl radical containing from 8 to 30 carbon atoms, for example tallow fatty acid derivatives, R₁₃ represents a hydrogen atom, a C₁-C₄ alkyl group or an alkenyl or alkyl group containing from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl sulfonates or alkylaryl sulfonates, the alkyl and aryl groups of which preferably comprise, respectively, from 1 to 20 carbon atoms and from 6 to 30 carbon atoms. R₁₂ and R₁₃ preferably denote a mixture of alkyl or alkenyl groups comprising from 12 to 21 carbon atoms, for example tallow fatty acid derivatives, R₁₄ denotes a methyl group, and R₁₅ denotes a hydrogen atom. Such a product is sold, for example, under the name Rewoquat® W 75 by the company Rewo;
quaternary diammonium or triammonium salts, in particular of formula (V):

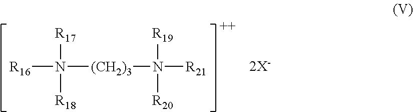

in which R₁₆ denotes an alkyl radical containing approximately from 16 to 30 carbon atoms, which is optionally hydroxylated and/or interrupted with one or more oxygen atoms, R₁₇ is chosen from hydrogen and an alkyl radical containing from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, are chosen from hydrogen and an alkyl radical containing from 1 to 4 carbon atoms, and X— is an anion chosen from the group of halides, acetates, phosphates, nitrates and methyl sulfates. Compounds of this kind are, for example, Finquat CT-P, available from the company Finetex (Quaternium 89), and Finquat CT, available from the company Finetex (Quaternium 75),
quaternary ammonium salts containing at least one ester function, such as those of formula (VI) below:

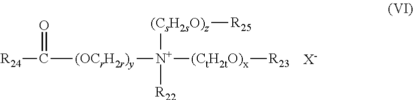

in which:

R₂₂ is chosen from C₁-C₆ alkyl groups and C₁-C₆ hydroxyalkyl or dihydroxyalkyl groups;

R₂₃ is chosen from:
the group

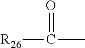

groups R₂₇ which are linear or branched, saturated or unsaturated C₁-C₂₂ hydrocarbon-based groups,
a hydrogen atom,

R₂₅ is chosen from:
the group

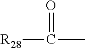

groups R₂₉ which are linear or branched, saturated or unsaturated C₁-C₆ hydrocarbon-based groups,
a hydrogen atom,

R₂₄, R₂₆ and R₂₈, which may be identical or different, are chosen from linear or branched, saturated or unsaturated C₇-C₂₁ hydrocarbon-based groups;

r, s and t, which may be identical or different, are integers ranging from 2 to 6;

r1 and t1, which may be identical or different, are equal to 0 or 1,

and r2+r1=2r and t1+t2=2t,

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₂₃ denotes R₂₇ and that when z is 0, then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and may contain from 12 to 22 carbon atoms, or may be short and may contain from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, have values of 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. It is possible, however, to use methanesulfonate, phosphate, nitrate or tosylate, an anion derived from organic acid, such as acetate or lactate, or any other anion that is compatible with ester-functional ammonium.

The anion X⁻ is even more particularly chloride or methyl sulfate.

Use is made more particularly, in the composition according to the invention, of the ammonium salts of formula (VI) in which:

R₂₂ denotes a methyl or ethyl group,

x and y are equal to 1;

z is equal to 0 or 1;

r, s and t are equal to 2;

R₂₃ is chosen from:
the group

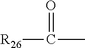

methyl, ethyl or C₁₄-C₂₂ hydrocarbon-based groups,
a hydrogen atom;

R₂₅ is chosen from:
the group

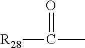

a hydrogen atom;

R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

The hydrocarbon-based groups are advantageously linear.

Mention may be made, for example, of the compounds of formula (VI) such as the diacyloxyethyldimethylammonium, diacyloxyethylhydroxyethylmethylammonium, monoacyloxyethyl-dihydroxyethylmethylammonium, triacyloxyethylmethylammonium and monoacyloxyethylhydroxyethyldimethylammonium salts (chloride or methyl sulfate in particular), and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, of triisopropanolamine, of alkyldiethanolamine or of alkyldiisopropanolamine, which are optionally alkoxylated, with C₁₀-C₃₀ fatty acids or with mixtures of C₁₀-C₃₀ fatty acids of plant or animal origin, or by transesterification of their methyl esters. This esterification is followed by quaternization using an alkylating agent such as an alkyl (preferably methyl or ethyl) halide, a dialkyl (preferably methyl or ethyl) sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts.

Mixtures of ammonium salts that may be used include, for example, the mixture containing 15% to 30% by weight of acyloxy-ethyldihydroxyethylmethylammonium methyl sulfate, 45% to 60% of diacyloxyethylhydroxyethylmethylammonium methyl sulfate and 15% to 30% of triacyloxyethylmethylammonium methyl sulfate, the acyl groups having from 14 to 18 carbon atoms and originating from palm oil, which is optionally partially hydrogenated.

It is also possible to use the ammonium salts containing at least one ester function that are described in U.S. Pat. No. 4,874,554 and U.S. Pat. No. 4,137,180.

Use may be made of behenoylhydroxypropyltrimethyl-ammonium chloride sold by KAO under the name Quatarmin BTC 131.

Preferably, the ammonium salts containing at least one ester function contain two ester functions.

Among the quaternary ammonium salts containing at least one ester function, which may be used, it is preferred to use dipalmitoylethylhydroxyethylmethylammonium salts.

When they are present, the amount of the cationic surfactant(s) is preferably in the range from 0.01% to 20% by weight and better still from 0.5% to 10% by weight, relative to the total weight of the composition.

Chelating agents that may be mentioned include diethylenetriaminepentaacetic acid and salts thereof.

The thickener(s) that may be present in composition (A) used according to the invention may be chosen from cellulose-based thickeners, for example hydroxyethylcellulose, hydroxypropylcellulose and carboxymethylcellulose, guar gum and derivatives thereof, for example the hydroxypropyl guar sold by the company Rhodia under the reference Jaguar HP 105, gums of microbial origin, such as xanthan gum and scleroglucan gum, synthetic thickeners such as crosslinked acrylic acid or acrylamidopropane-sulfonic acid homopolymers, for example Carbomer, nonionic, anionic, cationic or amphoteric associative polymers, such as the polymers sold under the names Pemulen TR1 or TR2 by the company Goodrich, Salcare SC90 by the company Ciba, Aculyn 22, 28, 33, 44 or 46 by the company Röhm & Haas, and Elfacos T210 and T212 by the company Akzo. Mention may also be made of thickening polymers of polysaccharide type.

According to one preferred embodiment, the reducing composition (A) may also comprise ammonium bicarbonate.

The pH of the reducing composition (A) preferably ranges between 7 and 14 and more preferentially from 8 to 10.

It may be adjusted to the desired value by means of acidifying or basifying agents usually used in the dyeing of keratin fibres, or alternatively using standard buffer systems.

Among the acidifying agents, examples that may be mentioned include mineral or organic acids, for instance hydrochloric acid, orthophosphoric acid, sulfuric acid and sulfonic acids, and carboxylic acids, for instance acetic acid, tartaric acid, citric acid or lactic acid.

Among the basifying agents that may be mentioned, for example, are aqueous ammonia, ammonium or alkali metal carbonates or bicarbonates, alkanolamines such as monoethanolamine, diethanolamine and triethanolamine and derivatives thereof, sodium hydroxide, potassium hydroxide and the compounds of formula (VII) below:

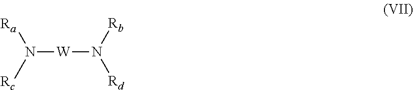

in which:

W is a propylene residue optionally substituted with a hydroxyl group or a C₁-C₄ alkyl radical;

Rₐ, R_{b}, R_{c} and R_{d}, which may be identical or different, represent a hydrogen atom or a C₁-C₄ alkyl or C₁-C₄ hydroxyalkyl group.

Aqueous ammonia and ammonium bicarbonate are particularly preferred as pH regulators.

The reducing composition (A) may be in liquid form or in thickened form. It may be applied from a heating bag, by brush or directly from the tube.

Generally, the reducing composition (A) is left to stand on the keratin fibres for 1 minute to 30 minutes and preferably for 1 minute to 10 minutes.

After the optional leave-on time, the hair is rinsed, preferably with water.

The oxidizing composition (B) used to reform the disulfide bonds and to attenuate the odour of the reduced keratin fibres generally comprises one or more oxidizing agents chosen from hydrogen peroxide, alkali metal bromates, polythionates, persalts such as perborates, percarbonates and persulfates, adsorbed or non-adsorbed metal salts, and enzymes of the 2-electron oxidase family.

Preferably, the oxidizing agent is hydrogen peroxide in the form of aqueous hydrogen peroxide solution, or alkali metal bromates.

Even more preferably, the oxidizing agent is hydrogen peroxide in the form of aqueous hydrogen peroxide solution.

The oxidizing agent(s) generally represent from 0.1% to 10% by weight and preferably from 1% to 3% by weight relative to the total weight of the oxidizing composition (B).

Preferably, when the oxidizing agent is hydrogen peroxide, the oxidizing composition used in the process according to the invention may contain one or more hydrogen peroxide stabilizers.

Mention may be made in particular of alkali metal or alkaline-earth metal pyrophosphates, such as tetrasodium pyrophosphate, alkali metal or alkaline-earth metal stannates, phenacetin or oxyquinoline acid salts, for instance oxyquinoline sulfate (8-hydroxyquinoline sulfate). Even more advantageously, use is made of at least one stannate optionally in combination with at least one pyrophosphate.

The hydrogen peroxide stabilizer(s) generally represent from 0.0001% to 5% by weight and preferably from 0.01% to 2% by weight relative to the total weight of the oxidizing composition.

For the purpose of improving the cosmetic properties of hair fibres or of attenuating or avoiding their degradation, the oxidizing composition used in the process according to the invention may also comprise one or more cosmetic active agents, such as those mentioned previously with regard to the reducing composition (A).

The pH of the oxidizing composition ranges from 1 to 13, preferably from 1.5 to 8 and better still from 1.5 to 5.

Preferably, the oxidizing composition (B) is left to stand on the fibres for 1 minute to 30 minutes and preferably for 1 minute to 15 minutes. After the optional leave-on time, the hair is rinsed, preferably with water.

The care composition (C) used in the treatment process according to the invention comprises one or more cationic polymers, which are preferably non-silicone polymers.

For the purposes of the present invention, the term “cationic polymer” denotes any polymer containing cationic groups and/or groups that can be ionized into cationic groups.

The cationic polymers that may be present in the composition according to the invention may be chosen from any of those already known per se as improving the cosmetic properties of the hair, i.e. especially those described in patent application EP-A-337 354 and in French patents FR-2 270 846, 2 383 660, 2 598 611, 2 470 596 and 2 519 863.

The polymer(s) are chosen from non-silicone polymers.

The cationic polymers that are preferred are chosen from those containing units comprising primary, secondary, tertiary and/or quaternary amine groups, which may either form part of the main polymer chain or be borne by a side substituent directly attached thereto.

The cationic polymers used in the care composition (C) generally have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type.

These are known products. They are described in particular in French patents 2 505 348 and 2 542 997. Among the said polymers, mention may be made of:

(1) Homopolymers or copolymers derived from acrylic or methacrylic esters or amides and comprising at least one of the units of formula (VIII), (IX), (X) or (XI) below:

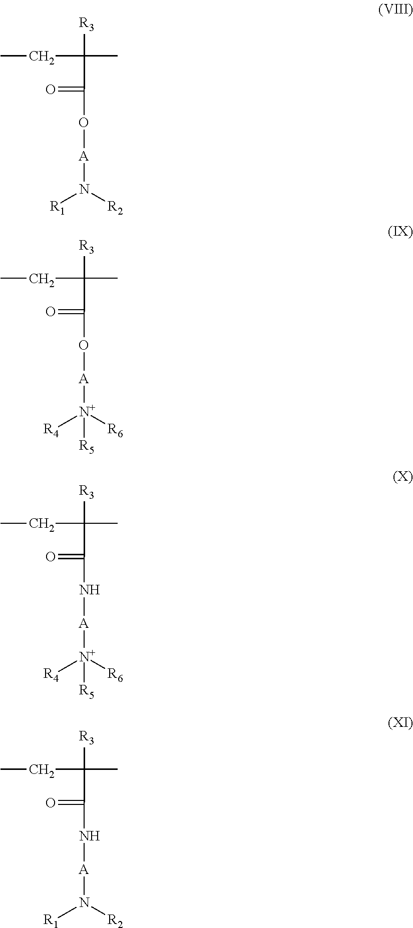

in which:

R₃, which may be identical or different, denotes a hydrogen atom or a CH₃ radical;

A, which may be identical or different, represents a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

R₁ and R₂, which may be identical or different, represent hydrogen or an alkyl group containing from 1 to 6 carbon atoms, and preferably methyl or ethyl;

X denotes an anion derived from a mineral or organic acid, such as a methosulfate anion or a halide such as chloride or bromide.

Mention may be made in particular of the ethyltrimethylammonium methacrylate chloride homopolymer.

The polymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these polymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyl-oxyethyltrimethylammonium chloride which are described, for example, in patent application EP-A-080976 and are sold under the name Bina Quat P 100 by the company Ciba Geigy,
the copolymer of acrylamide and of methacryloyl-oxyethyltrimethylammonium methosulfate sold under the name Reten by the company Hercules,
quaternized or non-quaternized vinylpyrrolidone/dialkyl-aminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinyl-pyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP, and
quaternized vinylpyrrolidone/dimethylaminopropylmethacryl-amide copolymers such as the product sold under the name Gafquat HS 100 by the company ISP, and
the crosslinked polymers of methacryloyloxy(C₁-C₄)alkyl tri(C₁-C₄)alkylammonium salts, such as the polymers obtained by homopolymerization of quaternized dimethylaminoethyl methacrylate with methyl chloride, or by copolymerization of acrylamide with dimethylaminoethyl methacrylate quaternized with methyl chloride, the homo- or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester may also be used. These dispersions are sold under the names Salcare® SC 95 and Salcare® SC 96 by the company Ciba.

(2) Cationic polysaccharide derivatives, including:

Cellulose ethers comprising quaternary ammonium groups, described in French patent 1 492 597, and in particular polymers sold under the names Ucare Polymer “JR” (JR 400, JR 125 and JR 30M) or “LR” (LR 400 or LR 30M) by the company Amerchol. These polymers are also defined in the CTFA dictionary as quaternary ammoniums of hydroxyethylcellulose that have reacted with an epoxide substituted with a trimethylammonium group.

Cellulose copolymers or cellulose derivatives grafted with a water-soluble quaternary ammonium monomer, and described especially in U.S. Pat. No. 4,131,576, such as hydroxyalkylcelluloses, for instance hydroxymethyl-, hydroxyethyl- or hydroxypropyl-celluloses grafted, in particular, with a methacryloylethyltrimethylammonium, methacrylamidopropyltrimethylammonium or dimethyl-diallylammonium salt.

The commercial products corresponding to this definition are more particularly the products sold under the names Celquat L 200 and Celquat H 100 by the company National Starch.

The cationic guar gums described more particularly in U.S. Pat. Nos. 3,589,578 and 4,031,307, such as guar gums containing cationic trialkylammonium groups. Guar gums modified with a 2,3-epoxypropyltrimethylammonium salt (e.g. chloride) are used, for example.

Such products are sold in particular under the trade names Jaguar C13S, Jaguar C 15, Jaguar C 17 or Jaguar C162 by the company Rhodia.

(3) Polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are described, in particular, in French patents 2 162 025 and 2 280 361.

(4) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bisunsaturated derivative, a bis-halohydrin, a bisazetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine function(s), they can be quaternized. Such polymers are described, in particular, in French patents 2 252 840 and 2 368 508.

(5) Polyamino amide derivatives resulting from the condensation of polyalkylene polyamines with polycarboxylic acids followed by alkylation with difunctional agents. Mention may be made, for example, of adipic acid/dialkylaminohydroxy-alkyldialkylenetriamine polymers in which the alkyl radical contains from 1 to 4 carbon atoms and preferably denotes methyl, ethyl or propyl. Such polymers are especially described in French patent 1 583 363.

Among these derivatives, mention may be made more particularly of the adipic acid/dimethylamino-hydroxy-propyl/diethylenetriamine polymers sold under the name Cartaretine F, F4 or F8 by the company Sandoz.

(6) Polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The mole ratio between the polyalkylene polyamine and the dicarboxylic acid is between 0.8:1 and 1.4:1; the polyamino amide resulting therefrom is reacted with epichlorohydrin in a mole ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(7) Cyclopolymers of alkyldiallylamine or of dialkyldiallylammonium, such as the homopolymers or copolymers containing, as main constituent of the chain, units corresponding to formula (XII) or (XIII):

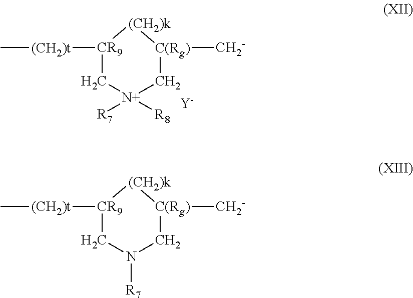

in which formulae k and t are equal to 0 or 1, the sum k+t being equal to 1; R₉ denotes a hydrogen atom or a methyl radical; R₇ and R₈, independently of each other, denote an alkyl group containing from 1 to 6 carbon atoms, a hydroxyalkyl group in which the alkyl group preferably contains 1 to 5 carbon atoms, or a lower (C₁-C₄) amidoalkyl group, or R₇ and R₈ may denote, together with the nitrogen atom to which they are attached, heterocyclic groups, such as piperidyl or morpholinyl; R₇ and R₈, independently of each other, preferably denote an alkyl group containing from 1 to 4 carbon atoms; Y⁻ is an anion such as bromide, chloride, acetate, borate, citrate, tartrate, bisulfate, bisulfite, sulfate or phosphate. These polymers are especially described in French patent 2 080 759 and in its Certificate of Addition 2 190 406.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the names Merquat 550 and Merquat 7SPR.

(8) The quaternary diammonium polymer containing repeating units corresponding to formula (XIV):

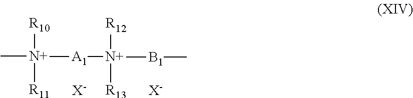

in which formula (XIV):

R₁₀, R₁₁, R₁₂ and R₁₃, which may be identical or different, represent aliphatic, alicyclic or arylaliphatic radicals containing from 1 to 6 carbon atoms or lower hydroxyalkylaliphatic radicals, or alternatively R₁₀, R₁₁, R₁₂ and R₁₃, together or separately, constitute, with the nitrogen atoms to which they are attached, heterocycles optionally containing a second heteroatom other than nitrogen, or alternatively R₁₀, R₁₁, R₁₂ and R₁₃ represent a linear or branched C₁-C₆ alkyl radical substituted with a nitrile, ester, acyl or amide group or a group COOR₁₄D or CONHR₁₄D where R₁₄ is an alkylene and D is a quaternary ammonium group;

A₁ and B₁ represent polymethylene groups containing from 2 to 8 carbon atoms, which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A₁, R₁₀ and R₁₂ can form, with the two nitrogen atoms to which they are attached, a piperazine ring; in addition, if A₁ denotes a linear or branched, saturated or unsaturated alkylene or hydroxyalkylene radical, B₁ can also denote a group —(CH₂)nCODOC(CH₂)n in which D denotes:

a) a glycol residue of formula: —O—Z—O—, in which Z denotes a linear or branched hydrocarbon-based radical or a group corresponding to one of the following formulae:

—(CH₂—CH₂—O)x-CH₂—CH₂

—[CH₂—CH(CH₃)—O]y—CH₂—CH(CH₃)—

in which x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

It is more particularly possible to use polymers that are formed from repeating units corresponding to formula (XV) below:

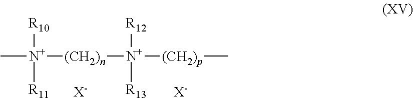

in which R₁₀, R₁₁, R₁₂ and R₁₃, which may be identical or different, denote an alkyl or hydroxyalkyl radical containing from 1 to 4 carbon atoms approximately, n and p are integers ranging from 2 to 8 approximately, and X⁻ is an anion derived from a mineral or organic acid. Mention may be made in particular of Mexomer PO sold by the company Chimex.

(9) Polyquaternary ammonium polymers formed from repeating units of formula (XVI):

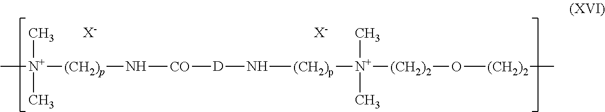

in which p denotes an integer ranging from 1 to 6 approximately, D may be nothing or may represent a group —(CH₂)ᵣ—CO— in which r denotes a number equal to 4 or 7, and X⁻ is an anion.

Such polymers may be prepared according to the processes described in U.S. Pat. Nos. 4,157,388, 4,702,906 and 4,719,282. They are especially described in Patent application EP-A-122 324.

Among these polymers, examples that may be mentioned include the products Mirapol A 15, Mirapol AD1, Mirapol AZ1 and Mirapol 175 sold by the company Miranol.

(10) Quaternary polymers of vinylpyrrolidone and of vinylimidazole, for instance the products sold under the names Luviquat FC 905, FC 550 and FC 370 by the company BASF. These polymers may also comprise other monomers, for instance diallyldialkylammonium halides. Mention may be made in particular of the product sold under the name Luviquat Sensation by the company BASF.

(11) Polyamines such as Polyquart H sold by Henkel, which is given under the reference name Polyethylene glycol (15) Tallow Polyamine in the CTFA dictionary, or oxyethylenated (15 OE) coconut polyamines.

Other cationic polymers that may be used in the context of the invention are polyalkyleneimines, in particular polyethyleneimines, polymers containing vinylpyridine or vinylpyridinium units, condensates of polyamines and of epichlorohydrin, polyquaternary ureylenes and chitin derivatives.

Among all the cationic polymers that may be used in the context of the present invention, it is preferred to use polymers of families (1), (2), (7), (8) and (10).

Preferably, the cationic polymer(s) used in the care composition (C) are chosen from cationic celluloses, cationic guar gums and quaternary polymers of vinylpyrrolidone and of vinylimidazole optionally combined with other monomers.

Even more preferentially, cationic polymer(s) used in the care composition (C) are chosen from cationic guar gums, in particular guar gums containing one or more trialkylammonium cationic groups, and especially a hydroxypropyltrimethylammonium group.

The cationic polymer(s) used in composition (C) according to the invention may be present in a content ranging from 0.05% to 5% by weight and preferably in a content ranging from 0.2% to 2% by weight, relative to the total weight of the care composition (C).

The care composition (C) according to the present invention also preferably contains one or more amino silicones (i).

The term “amino silicone” means any polyaminosiloxane, i.e. any polysiloxane comprising at least one primary, secondary or tertiary amine function or a quaternary ammonium group.

In the text hereinbelow, the term “silicone” is intended to denote, in accordance with what is generally accepted, any organosilicon polymer or oligomer of linear or cyclic, branched or crosslinked structure, of variable molecular weight, obtained by polymerization and/or polycondensation of suitably functionalized silanes, and consisting essentially of a repetition of main units in which the silicon atoms are linked together via oxygen atoms (siloxane bond —Si—O—Si—), optionally substituted hydrocarbon-based radicals being directly linked via a carbon atom to the said silicon atoms. The hydrocarbon-based radicals that are the most common are alkyl radicals, especially of C₁-C₁₀, and in particular methyl, fluoroalkyl radicals, the alkyl part of which is of C₁-C₁₀, and aryl radicals and in particular phenyl.

The amino silicones used in the care composition (C) according to the present invention are chosen from:

(a) the compounds corresponding to formula (XVII) below:

(R¹)ₐ(T)₃₋ₐ-Si[OSi(T)₂]ₙ—[OSi(T)_{b}(R¹)_{2-b}]ₘ—OSi(T)₃₋ₐ-(R¹)ₐ (XVII)

in which:

T is a hydrogen atom or a phenyl, hydroxyl (—OH) or C₁-C₈ alkyl radical, and preferably methyl, or a C₁-C₈ alkoxy, preferably methoxy,

a denotes the number 0 or an integer from 1 to 3, and preferably 0,

b denotes 0 or 1, and in particular 1,

m and n are numbers such that the sum (n+m) can range especially from 1 to 2000 and in particular from 50 to 150, it being possible for n to denote a number from 0 to 1999 and in particular from 49 to 149, and for m to denote a number from 1 to 2000 and in particular from 1 to 10;

R₁ is a monovalent radical of formula —C_{q}H_{2q}L in which q is a number from 2 to 8 and L is an amino group chosen from the following groups:

—N(R²)—CH₂—CH₂—N(R²)₂;

—N(R²)₂; —N⁺(R²)₃Q⁻;

—N⁺(R²)(H)₂Q⁻;

—N⁺(R²)₂HQ⁻;

—N(R²)—CH₂—CH₂—N⁺(R²)(H)₂Q⁻,

in which R² denotes a hydrogen atom, a phenyl, a benzyl or a saturated monovalent hydrocarbon-based radical, for example a C₁-C₂₀ alkyl radical, and Q⁻ represents a halide ion, for instance fluoride, chloride, bromide or iodide.

In particular, the amino silicones corresponding to the definition of formula (XVII) are chosen from the compounds corresponding to formula (XVIII) below:

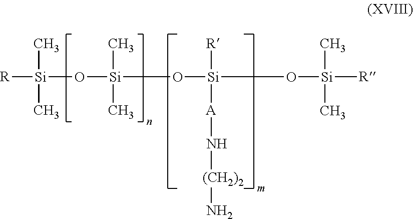

in which R, R′ and R″, which may be identical or different, denote a C₁-C₄ alkyl radical, preferably CH₃; a C₁-C₄ alkoxy radical, preferably methoxy; or OH; A represents a linear or branched, C₃-C₈ and preferably C₃-C₆ alkylene radical; m and n are integers that are dependent on the molecular weight and whose sum is between 1 and 2000.

According to a first possibility, R, R′ and R″, which may be identical or different, represent a C₁-C₄ alkyl or hydroxyl radical, A represents a C₃ alkylene radical and m and n are such that the weight-average molecular mass of the compound is between 5000 and 500 000 approximately. Compounds of this type are referred to in the CTFA dictionary as “amodimethicones”.

According to a second possibility, R, R′ and R″, which may be identical or different, represent a C₁-C₄ alkoxy or hydroxyl radical, at least one of the radicals R or R″ is an alkoxy radical and A represents a C₃ alkylene radical. The hydroxy/alkoxy mole ratio is preferably between 0.2/1 and 0.4/1 and advantageously equal to 0.3/1. Moreover, m and n are such that the weight-average molecular mass of the compound is between 2000 and 10⁶. More particularly, n is between 0 and 999 and m is between 1 and 1000, the sum of n and m being between 1 and 1000.

In this category of compounds, mention may be made, inter alia, of the product Belsil® ADM 652 sold by Wacker.

According to a third possibility, R and R″, which are different, represent a C₁-C₄ alkoxy or hydroxyl radical, at least one of the radicals R or R″ is an alkoxy radical, R′ represents a methyl radical and A represents a C₃ alkylene radical. The hydroxy/alkoxy mole ratio is preferably between 1/0.8 and 1/1.1 and advantageously equal to 1/0.95. Moreover, m and n are such that the weight-average molecular mass of the compound is between 2000 and 200 000. More particularly, n is between 0 and 999 and m is between 1 and 1000, the sum of n and m being between 1 and 1000.

More particularly, mention may be made of the product Fluid WR® 1300 sold by Wacker.

According to a fourth possibility, R and R″ represent a hydroxyl radical, R′ represents a methyl radical and A is a C₄-C₈ and preferably C₄ alkylene radical. Moreover, m and n are such that the weight-average molecular mass of the compound is between 2000 and 10⁶. More particularly, n is between 0 and 1999 and m is between 1 and 2000, the sum of n and m being between 1 and 2000.

A product of this type is especially sold under the name DC 28299 by Dow Corning.

Note that the molecular mass of these silicones is determined by gel permeation chromatography (ambient temperature, polystyrene standard; μ styragem columns; eluent THF; flow rate 1 mm/m; 200 μl of a solution containing 0.5% by weight of silicone in THF are injected, and detection is performed by refractometry and UV-metry).

A product corresponding to the definition of formula (VI) is in particular the polymer known in the CTFA dictionary (7^{th} edition, 1997) as “trimethylsilyl amodimethicone”, corresponding to formula (XIX) below:

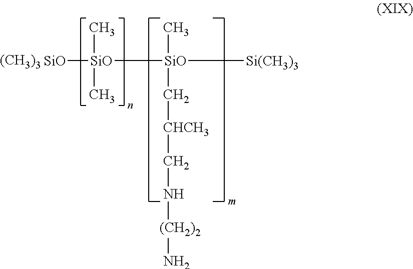

in which n and m have the meanings given above in accordance with formula (XVIII).

Such compounds are described, for example, in EP 0 095 238; a compound of formula (XIX) is sold, for example, under the name Q2-8220 by the company OSI.

(b) the compounds corresponding to formula (XX) below:

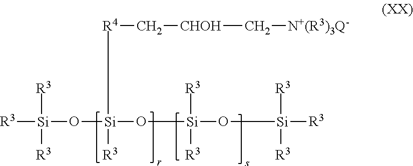

in which:

R³ represents a C₁-C₁₈ monovalent hydrocarbon-based radical, and in particular a C₁-C₈ alkyl or C₂-C₁₈ alkenyl radical, for example methyl;

R⁴ represents a divalent hydrocarbon-based radical, especially a C₁-C₈ alkylene radical or a divalent C₁-C₈, and for example C₁-C₈, alkylenoxy radical;

Q⁻ is a halide ion, in particular chloride;

r represents a mean statistical value from 2 to 20 and in particular from 2 to 8;

represents a mean statistical value from 20 to 200 and in particular from 20 to 50.

Such compounds are described more particularly in U.S. Pat. No. 4,185,087.

A compound falling within this class is the product sold by the company Union Carbide under the name Ucar Silicone ALE 56.

(c) the quaternary ammonium silicones of formula (XXI):

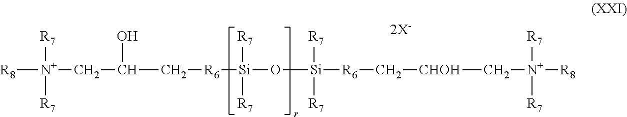

in which:

R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;

R₆ represents a divalent hydrocarbon-based radical, especially a C₁-C₁₈ alkylene radical or a divalent C₁-C₁₈, and for example C₁-C₈, alkylenoxy radical linked to the Si via an SiC bond;

R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;

X⁻ is an anion such as a halide ion, especially chloride, or an organic acid salt (acetate, etc.);

r represents a mean statistical value from 2 to 200 and in particular from 5 to 100;

These silicones are described, for example, in patent application EP-A 0 530 974.

d) the amino silicones of formula (XXII) below:

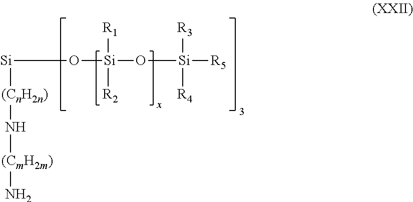

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and

in which x is chosen such that the amine number is between 0.01 and 1 meq/g.

Preferably, the amino silicone(s) (i) used in the care composition (C) are non-quaternized amino silicones.

For the purposes of the present invention, the term “non-quaternized amino silicone” means an amino silicone not comprising a permanent cationic charge, i.e. quaternized ammonium groups.

In other words, the non-quaternized amino silicone(s) comprise in their structure at least one primary, secondary or tertiary amine but do not comprise a quaternary ammonium function.

The amino silicone(s) (i) that are particularly preferred for use in the care composition (C) are polysiloxanes containing amine groups such as the silicones of formula (XVIII) or the silicones of formula (XIX).

When these compounds are used, one particularly advantageous embodiment involves their combined use with cationic and/or nonionic surfactants.

By way of example, use may be made of the product sold under the name Cationic Emulsion DC 929 by the company Dow Corning, which comprises, besides amodimethicone, a cationic surfactant comprising a mixture of products corresponding to formula (XXIII):

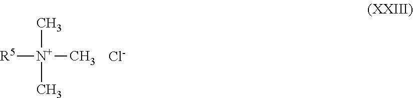

in which R⁵ denotes C₁₄-C₂₂ alkenyl and/or alkyl radicals derived from tallow fatty acids, and known under the CTFA name “tallowtrimonium chloride”, in combination with a nonionic surfactant of formula:

C₉H₁₉—C₆H₄—(OC₂H₄)₁₀—OH, known under the CTFA name “Nonoxynol 10”.

Use may also be made, for example, of the product sold under the name Cationic Emulsion DC 939 by the company Dow Corning, which comprises, besides amodimethicone, a cationic surfactant which is trimethylcetylammonium chloride and a nonionic surfactant of formula: C₁₃H₂₇—(OC₂H₄)₁₂—OH, known under the CTFA name “trideceth-12”.

Another commercial product that may be used according to the invention is the product sold under the name Dow Corning Q2 7224 by the company Dow Corning, comprising, in combination, the trimethylsilyl amodimethicone of formula (C) described above, a nonionic surfactant of formula: C₈H₁₇—C₆H₄—(OCH₂CH₂)₄₀OH, known under the CTFA name “octoxynol-40”, a second nonionic surfactant of formula: C₁₂H₂₅—(OCH₂—CH₂)₆—OH, known under the CTFA name “isolaureth-6”, and propylene glycol.

The silicone(s) (i) used in the care composition (C) according to the invention may be present in an amount ranging from 0.1% to 10% by weight, preferably in an amount ranging from 1% to 5% by weight and better still in a content ranging from 2% to 3% by weight relative to the total weight of the composition.

The pH of composition (C) ranges between 2 and 12, and preferably ranges from 2 to 8 and better still from 3 to 6.5. It may be adjusted with the same agents as for compositions (A) and (B).

One or more of the compositions (A), (B) and (C) according to the invention also comprise(s) one or more silicones (ii), different from the amino silicones (i), with a molecular weight of less than 10 000 and functionalized with one or more mercapto groups.

The functionalized silicone(s) (ii) used according to the invention are chosen from compounds having the following formulae:

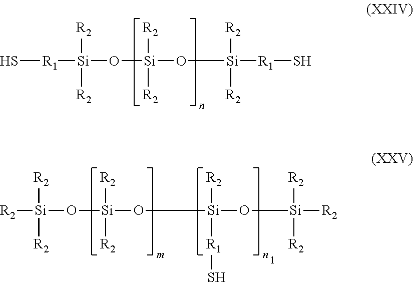

in which

R₁ denotes a saturated or unsaturated, linear or branched, optionally cyclic hydrocarbon-based chain comprising from 1 to 100 carbon atoms, optionally interrupted with a heteroatom chosen from N, O, S and P,

R₁ preferably denotes a C₁-C₁₀₀ alkylene group, better still a propylene group,

R₂ denotes an alkyl group containing from 1 to 6 carbon atoms or an alkoxy group containing from 1 to 6 carbon atoms,

R₂ preferably denotes a methyl group or a methoxy group,

n ranges from 0 to 132,

n₁ ranges from 1 to 132, and

m ranges from 1 to 132.

Preferably, the functionalized silicone(s) (ii) used in the present invention are chosen from the silicones of formula (XXIV).

As functionalized silicones (ii) used in the present invention, mention may be made of the mercaptosiloxane in which the mercapto functions are at the chain ends, sold by the company Shin-Etsu under the reference X-22-167B, and the mercaptosiloxane in which the mercapto functions are pendent, sold by the company Shin-Etsu under the reference KF-2001.

As explained previously, the functionalized silicone(s) (ii) may also be introduced into the reducing composition (A) and/or into the oxidizing composition (B) and/or into composition (C).

Thus, composition (A) or composition (B) or composition (C) comprises one or more silicones (ii) with a molecular weight of less than 10 000 and functionalized with one or more mercapto groups.

In one preferred variant of the invention, composition (C) comprises one or more functionalized silicones (ii). In other words, the functionalized silicone(s) (ii) are present only in composition (C).

Preferably, the functionalized silicone(s) (ii) are also introduced into at least one of the compositions (A) and/or (B).

Even more preferentially, the functionalized silicone(s) (ii) are present in compositions (A), (B) and (C).

The functionalized silicone(s) (ii) may be introduced into the composition(s) either in pure form or in the presence of one or more silicone-based or hydrocarbon-based solvents, or in the form of a latex.

The content of functionalized silicone(s) (ii) in the composition(s) containing them generally ranges from 0.5% to 20%, preferably from 0.5% to 5% and better still from 1% to 2% by weight relative to the total weight of the composition containing them.

For the purpose of improving the cosmetic properties of hair fibres or of attenuating or avoiding their degradation, the care composition (C) used in the process according to the invention may also comprise one or more cosmetic active agents, such as those mentioned previously with regard to the reducing composition (A) and the oxidizing composition (B).

In particular, the care composition (C) may comprise one or more non-siliceous fatty substances.

The composition according to the invention may also comprise one or more non-siliceous fatty substances.

The term “fatty substance” means an organic compound that is insoluble in water at standard temperature (25° C.) and at atmospheric pressure (760 mmHg, i.e. 1.013×10⁵ Pa), i.e. with a solubility of less than 5%, preferably of less than 1% and even more preferably of less than 0.1%. The non-siliceous fatty substances generally have in their structure a hydrocarbon-based chain comprising at least 6 carbon atoms and not comprising any siloxane groups. In addition, the fatty substances are generally soluble in organic solvents under the same temperature and pressure conditions, for instance chloroform, ethanol, benzene, liquid petroleum jelly or decamethylcyclopentasiloxane.

The term “non-siliceous fatty substance” means a fatty substance whose structure does not comprise any silicon atoms.

The fatty substances that may be used in the composition according to the invention are generally not oxyalkylenated and preferably do not contain any carboxylic acid COOH functions.

Preferably, the non-siliceous fatty substances of the invention are chosen from hydrocarbons, fatty alcohols, fatty esters, silicones and fatty ethers, and mixtures thereof.

Even more preferentially, they are chosen from hydrocarbons, fatty alcohols and fatty esters, and mixtures thereof.

They may be liquid or non-liquid, at room temperature and at atmospheric pressure.

The liquid fatty substances of the invention preferably have a viscosity of less than or equal to 2 Pa·s, better still less than or equal to 1 Pa·s and even better still less than or equal to 0.1 Pa·s at a temperature of 25° C. and at a shear rate of 1 s⁻¹.

The term “liquid hydrocarbon” means a hydrocarbon composed solely of carbon and hydrogen atoms, which is liquid at standard temperature (25° C.) and at atmospheric pressure (760 mmHg, i.e. 1.013×10⁵ Pa).

More particularly, the liquid hydrocarbons are chosen from:
linear or branched, optionally cyclic, C₆-C₁₆ alkanes. Examples that may be mentioned include hexane, undecane, dodecane, tridecane, and isoparaffins, for instance isohexadecane, isododecane and isodecane,
linear or branched hydrocarbons of mineral, animal or synthetic origin with more than 16 carbon atoms, such as volatile or non-volatile liquid paraffins and derivatives thereof, petroleum jelly, liquid petroleum jelly, polydecenes, hydrogenated polyisobutene such as the product sold under the brand name Parleam® by the company NOF Corporation, and squalane.

In one preferred variant, the liquid hydrocarbon(s) are chosen from volatile or non-volatile liquid paraffins, and derivatives thereof, and liquid petroleum jelly.

The term “liquid fatty alcohol” means a non-glycerolated and non-oxyalkylenated fatty alcohol, which is liquid at standard temperature (25° C.) and at atmospheric pressure (760 mmHg, i.e. 1.013×10⁵ Pa).

Preferably, the liquid fatty alcohols of the invention comprise from 8 to 30 carbon atoms.

The liquid fatty alcohols of the invention may be saturated or unsaturated.

The saturated liquid fatty alcohols are preferably branched. They may optionally comprise in their structure at least one aromatic or non-aromatic ring. They are preferably acyclic.

More particularly, the liquid saturated fatty alcohols of the invention are chosen from octyldodecanol, isostearyl alcohol and 2-hexyldecanol.

Octyldodecanol is most particularly preferred.

The unsaturated liquid fatty alcohols contain in their structure at least one double or triple bond, and preferably one or more double bonds. When several double bonds are present, there are preferably 2 or 3 of them, and they may be conjugated or unconjugated.

These unsaturated fatty alcohols may be linear or branched.

They may optionally comprise in their structure at least one aromatic or non-aromatic ring. They are preferably acyclic.

More particularly, the unsaturated liquid fatty alcohols of the invention are chosen from oleyl alcohol, linoleyl alcohol, linolenyl alcohol and undecylenyl alcohol.

Oleyl alcohol is most particularly preferred.

The term “liquid fatty esters” means an ester derived from a fatty acid and/or from a fatty alcohol that is liquid at standard temperature (25° C.) and at atmospheric pressure (760 mmHg, i.e. 1.013×10⁵ Pa).

The esters are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isodecyl neopentanoate and isostearyl neopentanoate.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy alcohols may also be used.

Mention may be made especially of: diethyl sebacate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

The composition may also comprise, as liquid fatty ester, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include saccharose, glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or unconjugated carbon-carbon double bonds.

The esters according to this variant may also be chosen from mono-, di-, tri- and tetraesters, and polyesters, and mixtures thereof.

These esters may be chosen, for example, from oleates, laurates, palmitates, myristates, behenates, cocoates, stearates, linoleates, linolenates, caprates and arachidonates, or mixtures thereof, such as, in particular, oleopalmitate, oleostearate or palmitostearate mixed esters.

More particularly, use is made of monoesters and diesters and in particular of sucrose, glucose or methylglucose mono- or dioleates, stearates, behenates, oleopalmitates, linoleates, linolenates or oleostearates.

An example that may be mentioned is the product sold under the name Glucate® DO by the company Amerchol, which is a methylglucose dioleate.

Finally, use may also be made of natural or synthetic glycerol esters of mono-, di- or triacids.

Among these, mention may be made of plant oils.

As oils of plant origin or synthetic triglycerides that may be used in the composition of the invention as liquid fatty esters, examples that may be mentioned include:
triglyceride oils of plant or synthetic origin, such as liquid fatty acid triglycerides containing from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, camellina oil, tamanu oil, babassu oil and pracaxi oil, caprylic/capric acid triglycerides, for instance those sold by the company Stéarineries Dubois or those sold under the names Miglyol® 810, 812 and 818 by the company Dynamit Nobel, jojoba oil and shea butter oil.

Liquid fatty esters derived from monoalcohols will preferably be used as esters according to the invention.

Isopropyl myristate and isopropyl palmitate are particularly preferred.

The liquid fatty ethers are chosen from liquid dialkyl ethers such as dicaprylyl ether.

The fatty substances of the invention may be liquid or non-liquid at room temperature (25° C.) and at atmospheric pressure (760 mmHg, i.e. 1.013×10⁵ Pa).

The term “non-liquid” preferably means a solid compound or a compound that has a viscosity of greater than 2 Pa·s at a temperature of 25° C. and at a shear rate of 1 s-1.

More particularly, the non-liquid fatty substances are chosen from fatty alcohols, fatty acid and/or fatty alcohol esters, non-silicone waxes and fatty ethers, which are non-liquid and preferably solid.

The non-liquid fatty alcohols that are suitable for use in the invention are more particularly chosen from saturated or unsaturated, linear or branched alcohols comprising from 8 to 30 carbon atoms. Mention may be made, for example, of cetyl alcohol, stearyl alcohol and a mixture thereof (cetylstearyl alcohol).

As regards the non-liquid esters of fatty acids and/or of fatty alcohols, mention may be made especially of solid esters derived from C₉-C₂₆ fatty acids and from C₉-C₂₆ fatty alcohols.

Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate and stearyl myristate, and hexyl stearate.

Still within the context of this variant, esters of C₄-C₂₂ dicarboxylic or tricarboxylic acids and of C₁-C₂₂ alcohols and esters of monocarboxylic, dicarboxylic or tricarboxylic acids and of C₂-C₂₆ dihydroxy, trihydroxy, tetrahydroxy or pentahydroxy alcohols may also be used.

Mention may be made especially of diethyl sebacate, diisopropyl sebacate, diisopropyl adipate, di-n-propyl adipate, dioctyl adipate and dioctyl maleate.

Among all the esters mentioned above, it is preferred to use myristyl, cetyl or stearyl palmitates, and alkyl myristates such as cetyl myristate, stearyl myristate and myristyl myristate.

The non-silicone wax(es) are chosen especially from carnauba wax, candelilla wax, esparto wax, paraffin wax, ozokerite, plant waxes, such as olive tree wax, rice wax, hydrogenated jojoba wax or absolute flower waxes, such as the blackcurrant blossom essential wax sold by Bertin (France), or animal waxes, such as beeswaxes or modified beeswaxes (cerabellina), and ceramides.

The ceramides or ceramide analogues, such as glycoceramides, that may be used in the compositions according to the invention are known per se and are natural or synthetic molecules that may correspond to the general formula (XXVI) below:

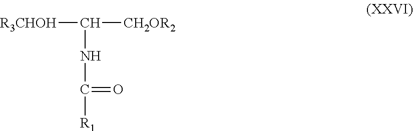

in which:
R₁ denotes a linear or branched, saturated or unsaturated alkyl radical, derived from C₁₄-C₃₀ fatty acids, this radical possibly being substituted with a hydroxyl group or a hydroxyl group in the omega position esterified with a saturated or unsaturated C₁₆-C₃₀ fatty acid;
R₂ denotes a hydrogen atom or a radical (glycosyl)n, (galactosyl)m or sulfogalactosyl, in which n is an integer ranging from 1 to 4 and m is an integer ranging from 1 to 8;
R₃ denotes a saturated or unsaturated C₁₅-C₂₆ hydrocarbon-based radical in the alpha position, this radical possibly being substituted with one or more C₁-C₁₄ alkyl radicals;

it being understood that, in the case of these natural ceramides or glycoceramides, R₃ may also denote a C₁₅-C₂₆ α-hydroxyalkyl radical, the hydroxyl group being optionally esterified with a C₁₆-C₃₀ α-hydroxy acid.

The ceramides that are preferred in the context of the present invention are those described by Downing in Arch. Dermatol., Vol. 123, 1381-1384, 1987, or those described in French patent FR 2 673 179.

The ceramide(s) that are more particularly preferred according to the invention are the compounds for which R₁ denotes a saturated or unsaturated alkyl derived from C₁₆-C₂₂ fatty acids; R₂ denotes a hydrogen atom and R₃ denotes a saturated linear C₁₅ radical.

Such compounds are, for example:
N-linoleyldihydrosphingosine,
N-oleyldihydrosphingosine,
N-palmitoyldihydrosphingosine,
N-stearoyldihydrosphingosine,
N-behenyldihydrosphingosine,

or mixtures of these compounds.

Even more preferentially, use is made of ceramides for which R₁ denotes a saturated or unsaturated alkyl radical derived from fatty acids; R₂ denotes a galactosyl or sulfogalactosyl radical; and R₃ denotes a group —CH═CH—(CH₂)₁₂—CH₃.

Other waxes or waxy starting materials that may be used according to the invention are especially marine waxes such as those sold by the company Sophim under the reference M82, and waxes of polyethylene or of polyolefins in general.

The non-liquid fatty ethers are chosen from dialkyl ethers and especially dicetyl ether and distearyl ether, alone or as a mixture.

Preferably, the non-siliceous fatty substances according to the invention are chosen from hydrocarbons, fatty alcohols, fatty esters and ceramides.

Even more preferably, the non-siliceous fatty substances are chosen from liquid petroleum jelly, stearyl alcohol, cetyl alcohol and a mixture thereof such as cetylstearyl alcohol, octyldodecanol, oleyl alcohol, isopropyl palmitate, isopropyl myristate, N-oleyldihydrosphingosine, N-behenyldihydrosphingosine and N-linoleyldihydrosphingosine.

It is also possible to use one or more fatty alcohols, which may be oxyalkylenated or glycerolated, in particular stearyl alcohol comprising 20 ethylene oxide groups.

The non-siliceous fatty substance(s) may be present in an amount ranging from 0.01% to 40% by weight and especially from 0.1% to 5% by weight relative to the total weight of the composition.

According to one preferred embodiment, the care composition (C) comprises one or more cationic polymers chosen from cationic guar gums, one or more amino silicones (i) chosen from the silicones of formulae (XVIII) and (XXI) and one or more functionalized silicones (ii) chosen from the silicones of formula (XXIV).

Preferably, composition (C) is left to stand on the fibres for 1 minute to 30 minutes and preferably for 5 minutes to 20 minutes. After the optional leave-on time, the hair may be rinsed, preferably with water.

Even more preferentially, the care composition (C) is not rinsed out.

The vehicle for the reducing composition (A), oxidizing composition (B) and care composition (C) used in the process according to the invention is preferably an aqueous medium formed by water and may advantageously contain one or more organic solvents, more particularly including non-fatty alcohols such as ethyl alcohol, isopropyl alcohol, benzyl alcohol and phenylethyl alcohol, for instance glycerol, ethylene glycol monomethyl, monoethyl and monobutyl ethers, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether. The organic solvents may then be present in concentrations of between about 0.1% and 20% and preferably between about 1% and 10% by weight relative to the total weight of the composition.

The reducing composition (A), the oxidizing composition (B) and the care composition (C) used in the process according to the invention may be, independently of each other, in the form of a lotion, a gel, a mousse, a cream or a paste.

As explained previously, the process according to the invention comprises a step of heating the hair to a temperature ranging from 60 to 250° C., after application of the care composition (C).

The heating step is necessary to optimize the effects of the process.

The step of heating the hair may be performed by drying the hair and/or by treating it with a heating appliance. In particular, the heating step may be performed using a hairdryer, a hood, heating rollers, a straightening iron, a curling iron, a crimping iron, a steam iron, an infrared lamp or an exothermic system in the composition. Preferably, the heating step is performed using a straightening iron.

Preferably, the step of heating the hair is performed at a temperature ranging from 80 to 220° C., preferably from 120 to 220° C. and better still from 120 to 200° C.

According to one particular embodiment, a pre-drying step is performed after the application of the care composition (C) and before the step of heating the hair performed at a temperature ranging from 60 to 250° C. The pre-drying step may be performed using a hairdryer.

According to one preferred embodiment, the cosmetic treatment process according to the invention comprises a step of applying a composition (A) comprising one or more sulfureous reducing agents to the keratin fibres, in particular to the hair, a rinsing step, a step of applying a composition (B) comprising one or more oxidizing agents, a rinsing step, a step of applying a rinse-out or leave-in care composition (C), comprising one or more cationic polymers, one or more amino silicones (i) and one or more silicones (ii) other than the amino silicones (i), with a molecular weight of less than 10 000 and functionalized with one or more mercapto groups, and a step of raising the temperature of the fibres to a temperature ranging from 60 to 250° C. and preferably from 80 to 220° C.

Preferably, compositions (A), (B) and (C) are applied successively to the keratin fibres, in particular the hair, at least one of the compositions (A) and/or (B) also comprising one or more silicones (ii) with a molecular weight of less than 10 000 and functionalized with one or more mercapto groups.

More preferentially, compositions (A), (B) and (C) are applied successively to the keratin fibres, in particular to the hair, compositions (A), (B) and (C) also comprising one or more silicones (ii) with a molecular weight of less than 10 000 and functionalized with one or more mercapto groups.

In other words, compositions (A), (B) and (C) of the process according to the invention each contain one or more silicones (ii) with a molecular weight of less than 10 000 and functionalized with one or more mercapto groups.

One particular process according to the invention consists in optionally applying one or more cosmetic treatments, especially a shampoo, after the step of rinsing out the reducing composition (A).

In particular, the care composition (C) is applied to the keratin fibres, which may then be straightened between the fingers to remove the excess product.

When it is desired to use the process of the invention for performing permanent-waving, mechanical means such as curlers are preferably used, the reducing composition being applied before, during or after the means for shaping the hair, preferably after.

Preferably, the reducing composition is applied to wet hair that has been rolled up beforehand on rollers that are from 2 to 30 mm in diameter. The composition may also be applied gradually as the hair is rolled up.

After applying the reducing composition (A), the head of hair may also be subjected to a heat treatment by heating to a temperature of between 30 and 250° C. throughout all or part of the leave-on time. In practice, this operation may be performed using a hairstyling hood, a hairdryer, a round or flat iron, an infrared ray dispenser or other standard heating appliances.

It is especially possible to use, both as a heating means and as a means for shaping the head of hair, an iron that heats to a temperature of between 60 and 220° C. and preferably between 120 and 200° C., the use of the heating iron taking place after the intermediate rinsing step following the application of the reducing composition.

The curler itself may be a heating means.

The oxidizing composition for reforming the disulfide bonds of keratin is then generally applied, to the rolled up or unrolled hair.

After removing the rollers, the head of hair is rinsed thoroughly, generally with water.

The step of heating the hair to a temperature ranging from 60 to 220° C. is performed as indicated previously.

When it is desired to uncurl or relax the hair, the reducing composition (A) is applied to the hair, and the hair is then subjected to mechanical reshaping for fixing the hair in its new shape, by means of a hair straightening operation, with a large-toothed comb, with the back of a comb, by hand or with a brush.

An oxidizing composition (B) for reforming the disulfide bonds of keratin is then generally applied to the rolled up or unrolled hair. After removing the rollers, the head of hair is rinsed thoroughly, generally with water.

The care composition (C) is then applied.

The step of heating the hair to a temperature ranging from 60 to 220° C. is performed as indicated previously.

A subject of the present invention is also a multi-compartment device or kit comprising:
a first compartment comprising a composition (A) comprising one or more sulfureous reducing agents,
a second compartment comprising an oxidizing composition (B) comprising one or more oxidizing agents, and
a third compartment comprising a care composition (C) comprising one or more cationic polymers and optionally one or more amino silicones (i),

at least one of the compositions (A), (B) and (C) comprising one or more silicones (ii) with a molecular weight of less than 10 000 and functionalized with one or more mercapto groups as defined previously.

In particular, the invention relates to a first multi-compartment device or kit comprising:
a first compartment comprising a composition (A) comprising one or more sulfureous reducing agents,
a second compartment comprising an oxidizing composition (B) comprising one or more oxidizing agents, and
a third compartment comprising a care composition (C) comprising one or more cationic polymers, one or more amino silicones (i) and one or more silicones (ii), other than the amino silicones (i), with a molecular weight of less than 10 000 and functionalized with one or more mercapto groups as defined previously.

In particular, the invention relates to a second multi-compartment device or kit comprising:
a first compartment comprising a reducing composition (A) comprising one or more sulfureous reducing agents and one or more silicones (ii) with a molecular weight of less than 10 000 and functionalized with one or more mercapto groups as defined previously,
a second compartment comprising an oxidizing composition (B) comprising one or more oxidizing agents, and
a third compartment comprising a care composition (C) comprising one or more cationic polymers, one or more amino silicones (i) and one or more silicones (ii), other than the amino silicones (i), with a molecular weight of less than 10 000 and functionalized with one or more mercapto groups as defined previously.

In particular, the invention relates to a third multi-compartment device or kit comprising:
a first compartment comprising a reducing composition (A) comprising one or more sulfureous reducing agents and one or more silicones (ii) with a molecular weight of less than 10 000 and functionalized with one or more mercapto groups as defined previously,
a second compartment comprising an oxidizing composition (B) comprising one or more oxidizing agents, one or more amino silicones (i) and one or more silicones (ii), other than the amino silicones (i), with a molecular weight of less than 10 000 and functionalized with one or more mercapto groups as defined previously,
a third compartment comprising a care composition (C) comprising one or more cationic polymers, one or more amino silicones (i) and one or more silicones (ii), other than the amino silicones (i), with a molecular weight of less than 10 000 and functionalized with one or more mercapto groups as defined previously.

The example that follows is given as an illustration of the present invention.

### EXAMPLE

1. Test Compositions

Compositions (A), (B) and (C) are prepared from the ingredients indicated in the table below, the amounts of which are expressed as weight percentages of active material relative to the total weight of the composition, unless otherwise indicated.

| | Composition | Composition | Composition |
|---|---|---|---|
| | (A) | (B) | (C) |
| | (reducing) | (oxidizing) | (care) |
| Cetearyl alcohol | — | — | 2.5% |
| Silicone | 4% | 4% | 4% |
| functionalized with | | | |
| mercapto groups | | | |
| (MW 10 000)⁽¹⁾ | | | |
| Fragrance | 0.3% | 0.2% | 0.3% |
| Thioglycolic acid | 3% | — | — |
| Ammonium | 2.7% | — | — |
| bicarbonate | | | |
| Pentasodium pentetate | 0.4% | — | — |
| Hydroxypropyl guar | — | — | 1 |
| trimethylammonium | | | |
| chloride⁽²⁾ | | | |
| H₂O₂ stabilizers | — | 0.28% | 0.7% |
| Oxyethylenated | — | — | 1.6% |
| (20 OE) stearyl | | | |
| alcohol⁽³⁾ | | | |
| Polydimethylsiloxane | — | — | 2% |
| comprising amine | | | |
| groups⁽⁴⁾ | | | |
| 50% hydrogen | — | 0.6% | — |
| peroxide solution | | | |
| pH agent qs | 8.2 | 3 | 5 |
| Water | 100 | 100 | 100 |

⁽¹⁾Mercaptosiloxane sold under the name X22-167B by the company Shin-Etsu
⁽²⁾Hydroxypropyl guar trimethylammonium sold under the name Jaguar C13S by the company Rhodia
⁽³⁾Oxyethylenated (20 OE) stearyl alcohol sold under the name Brij S20 PA by the company Uniqema
⁽⁴⁾Amino siloxane emulsion sold under the name Wacker Belsil ADM-LOG1 by the company Wacker

2. Application Process

Locks of frizzy sensitized hair are first washed and then towel-dried. The reducing composition (A) is then applied by brush and is left to stand on the locks for 10 minutes.

After rinsing the locks with water, the oxidizing composition (B) is applied and is left on the locks for one to two minutes.

The locks are rinsed with water and the care composition (C) is then applied carefully by brush. After a leave-on time of 15 minutes, the locks are straightened between the fingers to remove the excess product, and are then predried using a hairdryer with a flat brush up to 80% drying. An iron at a temperature of 200° C. is then applied.

Results

The treated locks have a smooth feel, both to the touch and visually. The locks do not have any frizziness.

It is also found that after 10 shampoo washes, the locks still feel very smooth and maintain reduced frizziness.

1-15. (canceled)

16. A cosmetic process for treating keratin fibers, comprising:
a) applying to the keratin fibers a reducing composition (A) comprising at least one sulfureous reducing agent;
b) rinsing the keratin fibers;
c) optionally applying to the keratin fibers an oxidizing composition (B) comprising at least one oxidizing agent and then rinsing;
d) applying a care composition (C) comprising at least one cationic polymer;
e) optionally rinsing the keratin fibers; and
f) heating the keratin fibers to a temperature ranging from about 60° C. to about 250° C. after applying the care composition (C);
wherein at least one of the compositions (A), (B) and (C) further comprises at least one silicone (ii) with a molecular weight of less than about 10,000 and functionalized with at least one mercapto group.

17. The process according to claim 16, wherein the keratin fibers are hair.

18. The process according to claim 16, wherein the at least one silicone (ii) is chosen from compounds having the following formulae: 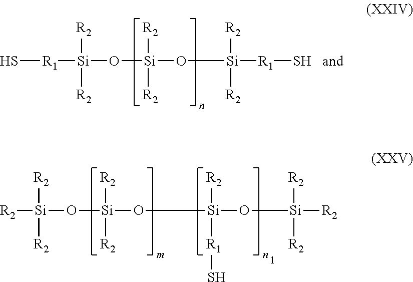
in which:
R₁ is a saturated or unsaturated, linear or branched, optionally cyclic hydrocarbon-based chain comprising from 1 to 100 carbon atoms, optionally interrupted with a heteroatom chosen from N, O, S and P;
R₂ is chosen from alkyl groups containing from 1 to 6 carbon atoms and alkoxy groups containing from 1 to 6 carbon atoms;
n ranges from 0 to 132;
n₁ ranges from 1 to 132; and
m ranges from 1 to 132.

19. The process according to claim 16, wherein the amount of the at least one silicone (ii) in composition (A), (B) and/or (C) ranges from about 0.5% to about 20% by weight, relative to the total weight of the composition containing it.

20. The process according to claim 19, wherein the amount of the at least one silicone (ii) in composition (A), (B) and/or (C) ranges from about 0.5% to about 5% by weight, relative to the total weight of the composition containing it.

21. The process according to claim 19, wherein the amount of the at least one silicone (ii) in composition (A), (B) and/or (C) ranges from about 1% to about 2% by weight, relative to the total weight of the composition containing it.

22. The process according to claim 16, wherein composition (C) comprises at least one amino silicone (i).

23. The process according to claim 22, wherein the at least one amino silicone (i) is chosen from:
(a) the compounds corresponding to formula (XVII) below:
(R¹)a(T)₃₋ₐ-Si[OSi(T)₂]ₙ—[OSi(T)_{b}(R¹)_{2-b}]ₘ—OSi(T)₃₋ₐ-(R¹)ₐ (XVII)
wherein:
T is chosen from hydrogen atoms, phenyl groups, hydroxyl (—OH) radicals, C₁-C₈ alkyl radicals, and C₁-C₈ alkoxy radicals;
a is 0 or an integer from 1 to 3;
b is 0 or 1;
m and n are numbers such that the sum (n+m) can range from 1 to 2000, it being possible for n to be a number from 0 to 1999, and for m to be a number from 1 to 2000; and
R¹ is a monovalent radical of formula —C_{q}H_{2q}L in which q is a number from 2 to 8 and L is an amino group chosen from the groups:
—N(R²)—CH₂—CH₂—N(R²)₂;
—N(R²)₂; —N⁺(R²)₃Q⁻;
—N⁺(R²)(H)₂Q⁻;
—N⁺(R²)₂HQ⁻; and
—N(R²)—CH₂—CH₂—N⁺(R²)(H)₂Q⁻, wherein R² is chosen from hydrogen, phenyl radicals, benzyl radicals and saturated monovalent hydrocarbon-based radicals, and Q⁻ is chosen from halide ions;
(b) the compounds corresponding to formula (XX) below: 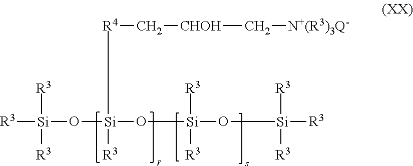
wherein:
R³ represents a C₁-C₁₈ monovalent hydrocarbon-based radical;
R⁴ represents a divalent hydrocarbon-based radical;
Q⁻ is a halide ion;
r represents a mean statistical value from 2 to 20; and
s represents a mean statistical value from 20 to 200,
(c) the quaternary ammonium silicones of formula (XXI): 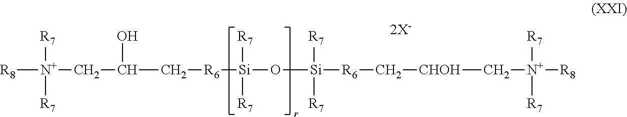
wherein:
R₇, which may be identical or different, represents a monovalent hydrocarbon-based radical containing from 1 to 18 carbon atoms;
R₆ represents a divalent hydrocarbon-based radical;
R₈, which may be identical or different, is chosen from hydrogen atoms, monovalent hydrocarbon-based radicals containing from 1 to 18 carbon atoms, C₂-C₁₈ alkenyl radicals, and radicals —R₆—NHCOR₇;
X⁻ is chosen from anions and organic acid salts; and
r represents a mean statistical value from 2 to 200; and
d) the amino silicones of formula (XXII) below: 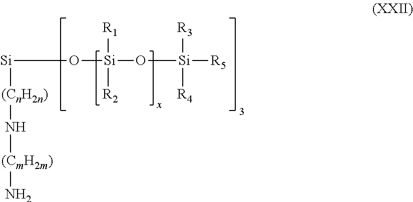
wherein:
R₁, R₂, R₃ and R₄, which may be identical or different, are chosen from C₁-C₄ alkyl radicals and phenyl groups,
R₅ is chosen from C₁-C₄ alkyl radicals and hydroxyl groups,
n is an integer ranging from 1 to 5, and
m is an integer ranging from 1 to 5, and
wherein x is chosen such that the amine number ranges from about 0.01 to about 1 meq/g.

24. The process according to claim 23, wherein the amino silicone corresponding to formula (XVII) is chosen from the compounds corresponding to formula (XVIII) below: 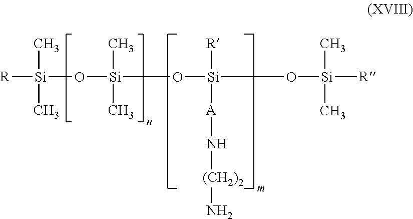
wherein
R, R′ and R″, which may be identical or different, are chosen from C₁-C₄ alkyl radicals; C₁-C₄ alkoxy radicals; and OH radicals;
A represents a linear or branched, C₃-C₈ alkylene radical; and
m and n are integers dependent on the molecular weight and whose sum is between 1 and 2000.

25. The process according to claim 23, wherein the amino silicone of formula (XVII) is trimethylsilyl amodimethicone, corresponding to formula (XIX) below: 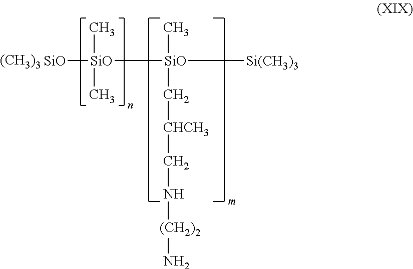
wherein m and n are integers that are dependent on the molecular weight and whose sum is between 1 and 2000.

26. The process according to claim 16, wherein the at least one cationic polymer is chosen from cationic celluloses, cationic guar gums and quaternary polymers of vinylpyrrolidone and of vinylimidazole optionally combined with other monomers.

27. The process according to claim 16, wherein composition (A) comprises at least one sulfureous reducing agent chosen from thiol-based and non-thiol-based reducing agents.

28. The process according to claim 27, wherein the thiol-based reducing agents are chosen from thioglycolic acid, thiolactic acid, cysteine, homocysteine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiopropionic acid, lipoic acid and N-acetylcysteine.

29. The process according to claim 28, wherein the thiol-based reducing agents are chosen from thioglycolic acid and thiolactic acid.

30. The process according to claim 27, wherein the non-thiol-based reducing agents are chosen from alkali metal and alkaline-earth metal sulfites.

31. The process according to claim 16, wherein the oxidizing composition (B) comprises at least one oxidizing agent chosen from hydrogen peroxide, alkali metal bromates, polythionates, persalts, adsorbed or non-adsorbed metal salts, and enzymes of the 2-electron oxidase family.

32. The process according to claim 31, wherein the at least one oxidizing agent is hydrogen peroxide.

33. The process according to claim 16, wherein heating the keratin fibers comprises heating at a temperature ranging from about 80 to about 220° C.

34. A multi-compartment device or kit comprising:
a first compartment comprising a composition (A) comprising at least one sulfureous reducing agent,
a second compartment comprising an oxidizing composition (B) comprising at least one oxidizing agent, and
a third compartment comprising a care composition (C) comprising at least one cationic polymer and optionally at least one amino silicone (i),
wherein at least one of the compositions (A), (B) and (C) further comprises at least one silicone (ii) with a molecular weight of less than about 10,000 and functionalized with at least one mercapto group.
